(19) United States
(12) Patent Application Publication
(10) Pub. No.: US 2016/0100934 A1
(43) Pub. Date: Apr. 14, 2016

(54) NANOMATERIALS FOR THE INTEGRATION OF SOFT INTO HARD TISSUE

(71) Applicant: NORTHEASTERN UNIVERSITY, Boston, MA (US)

(72) Inventors: Daniel J. Hickey, Boston, MA (US); Thomas J. Webster, Barrington, RI (US); Batur Ercan, Cambridge, MA (US)

(21) Appl. No.: 14/893,242

(22) PCT Filed: May 27, 2014
(86) PCT No.: PCT/US14/39577

§ 371 Date: Nov. 23, 2015

Related U.S. Application Data
(60) Provisional application No. 61/827,164, filed on May 24, 2013.

(51) Int. Cl. A61F 2/08; A61L 27/54; A61L 27/44; A61L 27/58; A61L 27/46

(52) U.S. Cl. CPC A61F 2/0811; A61L 27/58; A61L 27/46; A61L 27/446; A61L 27/54; A61L 2300/112; A61L 2300/102; A61L 2300/222; A61L 2300/64; A61L 2300/414; A61L 2430/10

(57) ABSTRACT

Nanocomposite materials are provided for attaching soft tissue to hard tissue of a mammalian subject. The materials include a biodegradable polymer network suffused with mineral nanoparticles. The nanocomposite materials have a surface structure that promotes the infiltration, adhesion and proliferation of cells such as osteoblasts and fibroblasts, and are useful to reconstruct enthesis tissue, such as a tendon bone insertion. Devices containing the nanocomposites and methods for implantation of the devices at a tendon-bone interface or ligament-bone interface are provided for reconstructive surgery.

### CROSS REFERENCE TO RELATED APPLICATIONS

This application claims the priority of U.S. Provisional Application No. 61/827,164 filed 24 May 2013 and entitled “Nanomaterials for the Integration of Soft into Hard Tissue”, the whole of which is hereby incorporated by reference.

### STATEMENT REGARDING FEDERALLY SPONSORED RESEARCH OR DEVELOPMENT

The invention was developed with financial support from Grant No. 0965843 from the National Science Foundation. The U.S. Government has certain rights in the invention.

### BACKGROUND

Biomaterials for the regeneration of bone tissue have been investigated for use in treatment of a growing population of people with damaged and degrading bone. While successful regeneration of bone defects has been demonstrated, there is considerable room for improvement. Ideally, biomaterials for tissue regeneration should mechanically match their implant location and possess suitable chemical and topographical properties for promoting cellular adhesion, proliferation, migration, and the secretion of extracellular matrix (ECM)-forming proteins. Calcium phosphate-based ceramics and composites have found the most widespread application for bone due to their mechanical properties and osteogenic capacity.

The enthesis is a region of tissue that forms an attachment of tendon or ligament to bone, provides support, and disperses stress concentrations at the site of insertion into bone. The structure of an enthesis is graded from highly mineralized fibrocartilage at the bone interface to un-mineralized fibrocartilage where it connects to the ligament or tendon. A ligament is a structure that connects two bones at a joint, and a tendon is a structure that attaches a muscle to a bone.

The enthesis lacks a direct blood supply and cannot regenerate once it is injured. It is believed that the loss of enthesis functionality following joint reconstruction surgery is a major factor contributing to the 5-25% failure rate of the 100,000 reconstructive surgeries performed on the anterior cruciate ligament (ACL) every year in the United States (Smith L, Thomopoulos S, US Muscoskeletal Rev., 6 (2011), 11-5). Therefore, there remains a need to develop an artificial, bioengineered construct or device that is capable of regenerating the critical tendon-to-bone or ligament-to-bone insertion site.

### SUMMARY OF THE INVENTION

The present invention provides composite materials and devices incorporating the materials for the attachment of soft tissue, such as a tendon or ligament, to hard tissue, such as bone. The composite material includes a polymer matrix that contains one or more types of mineral nanoparticles embedded within the polymer matrix. The matrix is preferably a biodegradable, hydrophilic polymer, such as poly (L-lactic acid) (PLLA). The mineral nanoparticles preferably contain or consist of magnesium oxide (MgO), hydroxyapatite (HA) or mixtures thereof. For example, MgO nanoparticles, HA nanoparticles, or a mixture of MgO nanoparticles and HA nanoparticles can be used. The nanoparticles preferably are mixed with a polymer material as a fluid suspension, which is then formed into a desired shape to produce a nanocomposite material. The nanocomposite material is then incorporated into a device, which is implanted in a surgical procedure to form a junction between soft tissue and hard tissue, where it leads to the generation of enthesis tissue at the junction over time.

One type of device provided by the invention is a tissue scaffold or enthesis regeneration device that can be used to regenerate an enthesis at a site of attachment of tendon or ligament to bone. To restore functionality to the enthesis following surgery, a ring-shaped scaffold device (enthesis device) is provided for implantation during surgery. The device cushions the insertion of ligament or tendon to bone and promotes generation or regeneration of an enthesis. The device requires minimal changes to current surgical procedures and promotes regeneration of the graded transition of the natural tendon bone insertion (TBI), thereby improving joint stability and increasing the success rate of reconstructive joint surgery. The device is preferably ring-shaped (similar to the shape of an “O-ring”) to fit snugly in a drilled bone tunnel and to allow orthopedic soft tissue to pass through the center of the device.

Thus, one aspect of the invention is a composite material comprising a polymer matrix and a plurality of nanoparticulate mineral particles embedded in the matrix. The polymer is preferably a biodegradable hydrophilic polymer that forms a matrix or surface suitable for infiltration or adhesion by cells of a mammal, such as a human subject, into which it is implanted. For example, the polymer can include or consist of PLLA, poly(lactic-co-glycolic acid) (PLGA), polycaprolactone (PCL), or collagen. The mineral nanoparticles can include or consist of MgO, magnesium phosphate (monobasic (Mg(H₂PO₄)₂), dibasic (MgHPO₄), tribasic (Mg₃(PO₄)₂), or any combination thereof), or another biocompatible mineral substance capable of releasing Mg²⁺ ions, HA, or any combination thereof having combined components admixed into a single population of nanoparticles (i.e., nanoparticles of heterogeneous composition) or present in separate populations of nanoparticles (i.e., nanoparticles of homogenous composition). The mineral nanoparticles are each present in the nanocomposite material at a level of from 0 to about 30 percent by weight of the dry ingredients used to form the nanocomposite, such as at a level of about 5 wt %, 10 wt %, 15 wt %, 20 wt %, 25 wt %, or 30 wt %. The mineral nanoparticles also can be present in the nanocomposite as a gradient of concentration of one or more mineral components or nanoparticles through the solid nanocomposite material.

Another aspect of the invention is an enthesis regeneration device containing the nanocomposite material described above, which is formed into a ring-shaped structure having an inner surface and an outer surface. The inner surface of the ring is configured to surround a tendon, ligament, or cell scaffold that is to be attached to a bone material, and the outer surface is configured to fit within a bone cavity. The bone cavity can be either naturally occurring in the bone or can be artificially produced, such as by drilling or cutting during a surgical procedure. In certain embodiments, the nanocomposite material contains a gradient of mineral nanoparticles, such as MgO and/or HA nanoparticles, from a higher concentration at one or more surfaces of the device intended to contact bone when surgically implanted to a lower concentration at one or more surfaces intended to contact soft tissue (i.e., tendon, ligament, or a cell scaffold) when surgically implanted. For example, the gradient can be from 0% at soft tissue contact to 30 wt % at bone contact, or from 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, or 20% to 30% (all percentages by weight of dry ingredients) of MgO nanoparticles, magnesium phosphate nanoparticles, HA nanoparticles, or combinations thereof (with concentrations independently varied). In certain embodiments of the device, the inside diameter is selected to provide contact of the device with a particular tendon, ligament, or cell scaffold and the outside diameter is selected to provide contact with an inner surface of a bone cavity. Thus, the dimensions of the device can be specifically tailored to match the anatomical requirements of a specific ligament or tendon, or a specific patient. For example, the device can be configured for use in reattachment or replacement of an anterior cruciate ligament of a human patient. In such an application, the dimensions of the ring-shaped device can include, for example, an inner diameter of about 10 mm, an outer diameter of about 14 mm, and a wall thickness of about 2 mm.

Yet another aspect of the invention is a method of method of fabricating a nanocomposite material as described above. The method includes the steps of: (a) providing a solution of a biocompatible polymer, such as poly(L-lactic acid), in a solvent; (b) suspending mineral nanoparticles, such as MgO nanoparticles, in the solution to form a suspension; (c) placing the suspension in a mold; and (d) removing the solvent to form the nanocomposite material. In certain embodiments of the method, the nanocomposite contains both MgO and HA nanoparticles. In certain embodiments of the method, the nanocomposite contains a gradient of MgO and HA nanoparticles. In certain embodiments, the suspension is placed into the mold by injection molding or by pouring. In certain embodiments, the solvent is removed by heating or by evaporation. In embodiments, the mold produces a sheet of material, or a material having a desired shape for use in reconstructive surgery, such as a ring shape.

Still another aspect of the invention is a method of attaching a ligament or tendon to a bone. The method includes the steps of: (a) attaching the enthesis regeneration device described above to a detached end of a ligament or tendon, whereby the device surrounds the detached end; (b) anchoring the detached end of the ligament or tendon within a hole or tunnel in a bone of an animal, such as a mammal or a human subject; and (c) allowing the device to form a new enthesis between the bone and the ligament or tendon.

Another aspect of the invention is a method of attaching a soft tissue to a hard tissue. The method includes the steps of: (a) attaching a device comprising the nanocomposite material described above to a soft tissue; (b) anchoring the device and attached soft tissue to a hard tissue; and (c) allowing cell attachment and proliferation within the device to attach the soft tissue to the hard tissue.

Still another aspect of the invention is a kit containing the enthesis regeneration device described above and a container for the device. The kit can optionally include instructions for using the device in a surgical reconstruction procedure. In some embodiments, the kit further includes one or more tools to aid in attaching the device to a tissue in a reconstructive surgery.

### BRIEF DESCRIPTION OF THE DRAWINGS

FIG. 1 shows a schematic representation of an embodiment of an enthesis regeneration device of the present invention. The device is a ring-shaped polymer matrix embedded with mineral nanoparticles. Two rings are placed around the ends of a ligament, which is then fitted into two adjacent bones at a joint. The right side of the diagram shows the location in the joint where the device is implanted. The bottom left depicts a cutout segment of the device having a graded mineralization of the polymer matrix with mineral nanoparticles between the bone- and ligament-contacting surfaces of the ring.

FIG. 2A shows a transmission electron micrograph of hydroxyapatite nanoparticles. FIG. 2B shows a transmission electron micrograph of MgO nanoparticles.

FIG. 3A shows a Fourier transform infrared (FTIR) spectrum of hydroxyapatite nanoparticles. FIG. 3B shows an X-ray diffraction analysis of MgO nanoparticles. FIG. 3C shows an FTIR spectrum of MgO nanoparticles.

FIGS. 4A through 4J show atomic force micrographs of the indicated surfaces. FIGS. 4K and 4L show the rms surface roughness obtained by AFM for the indicated surfaces obtained from 2 μm×2 μm (FIG. 4K) or 40 μm×40 μm (FIG. 4L) scans.

FIGS. 5A through 5D show scanning electron micrographs (SEMs) of PLLA and the indicated nanocomposite surfaces.

FIGS. 6A through 6F show the results of mechanical testing of PLLA and the indicated nanocomposite materials.

FIG. 7 shows stress-strain curves for PLLA and the indicated nanocomposite materials.

FIG. 8 shows SEMs of fracture planes of the indicated materials.

FIG. 9 shows the effect of mineral nanoparticles on bacterial growth. The growth of Staphylococcus aureus in tryptic soy broth (TSB) with the indicated nanoparticle concentrations was measured via absorbance at 562 nm at 2 min intervals. MgO nanoparticles completely inhibited bacterial growth.

FIG. 10A shows the adherence of osteoblasts to polymer material (PLLA) and mineralized polymer composite materials (PLLA containing hydroxyaspatite (HA) and/or MgO) after four hours. FIG. 10B shows results of a longer term adherence study (1-5 days) reflecting both cell adhesion and cell proliferation.

FIG. 11 shows the results of an experiment to measure adhesion of osteoblasts and fibroblasts on polymer composite materials containing PLLA polymer and nanoparticles of MgO and/or hydroxyapatite (HA). The number of adhered cells at four hours after seeding is represented.

FIG. 12 shows the results of a study of the effect of degradation of the indicated materials on pH.

### DETAILED DESCRIPTION OF THE INVENTION

The invention provides nanocomposite materials for use in attaching a soft tissue, such as a tendon or ligament, to a hard tissue, such as bone, during reconstructive or orthopedic surgery. The materials contain a matrix or scaffold of a polymer material that has been mineralized by the addition of a plurality of one or more types of nanoparticulate mineral materials. The nanocomposite materials are used to fabricate surgical devices that promote the regeneration of enthesis tissue at the insertion point of tendon or ligament into bone. The mineralization within the device promotes the adhesion and proliferation of osteoblasts, fibroblasts, and other cells, and also serves as a mechanical transition from soft to hard tissue.

A preferred polymer for the scaffold is poly(L-lactic acid) (PLLA), which effectively mimics the nanostructured extra-cellular matrix of the body. PLLA provides a three-dimensional cell-matrix network structure that is superior to many other biodegradable polymers for cell infiltration and attachment. In alternative embodiments, the biodegradable polymer can be, for example, poly(lactic-co-glycolic acid) (PLGA), poly(caprolactone) (PCL), polyhydroxybutyrate polypropylene fumarate, or a protein such as collagen or fibrin. The polymer can be incorporated into the material as a pre-polymerized polymer, such as by dissolving the material in a solvent and adding mineral nanoparticles to the solution, or by polymerizing the polymer from suitable precursors in the presence of the nanoparticles, e.g., by polymerizing the polymer in a suspension of nanoparticles in a suitable solvent. Yet another method that can be used to form the nanocomposite material is to: melt the polymer, if it can be melted without degrading; add nanoparticles to the melted polymer; optionally deposit the melted polymer-nanoparticle suspension into a mold; and allow the composite to cool in the mold. The polymer optionally can be cross-linked.

The nanocomposite material is preferably mineralized with magnesium oxide (MgO) nanoparticies to increase cell attachment and proliferation, as well as bone and ligament growth. MgO mineralization may be graded from highly mineralized at the bone interface to less mineralized or unmineralized at the tendon graft interface with the device. While the use of nanostructured MgO as a polymer additive is believed to be novel, its addition to a polymer scaffold has been found by the inventors to significantly increase cell attachment and proliferation, leading to bone and ligament growth. Other mineral nanoparticles, such as magnesium phosphate and hydroxyapatite (HA) nanoparticles, can be added to the material instead of or in addition to MgO nanoparticles; however, preferred embodiments include MgO nanoparticles, either alone or in combination with HA or other nanoparticles.

Magnesium (Mg) is a biocompatible, biodegradable, low-cost and environmentally friendly material that exists naturally in the human body. In bone, where Mg exists in its highest concentrations, Mg cations (Mg²⁺) reside along the edges of nanostructured bone apatite minerals (equivalent to hydroxyapatite) to directly influence mineral size and density—important factors which contribute to the unique mechanical properties of bone. Further, these Mg²⁺ ions indirectly influence mineral metabolism through activation of alkaline phosphatase. Beyond their cooperative role with hydroxyapatite (HA) in bone, Mg²⁺ ions play a key role in the functions of all cells in the body, specifically through their activation of integrins, which are cell surface receptors that mediate the interactions of cells with their extracellular environment. Divalent Mg²⁺ ions, as well as Ca²⁺ ions, initiate conformational activation of integrins for ligand binding by attaching to sites on the integrin α-chain, thereby influencing cell functions such as attachment, proliferation, and migration. Thus, integration of magnesium within tissue engineering constructs in the present invention serves to improve cell-scaffold interactions.

In the present invention, magnesium oxide (MgO) nanoparticles (which release small amounts of Mg²⁺ ions under physiological conditions) are dispersed within poly(L-lactic) acid (PLLA) polymer sheets, both alone and in combination with hydroxyapatite (HA) nanoparticles. The inventors examined the functional influence of Mg within the body and its application in tissue engineering, in which fabricated scaffolds are used to mimic the body's extracellular matrix (ECM). Bulk Mg is biodegradable and stiffness and strength similar to bone. Weng and Webster demonstrated that bone cell density increases on nanostructured Mg compared to unmodified bulk Mg. Weng, L., and T. J. Webster, Nanostructured Magnesium Increases Bone Cell Density, Nanotechnology, 23, 2012. However, Mg has found limited application clinically because of its fast degradation kinetics under physiological conditions. Mg is known to release Mg²⁺ ions, OH⁻ ions, and hydrogen (H₂) gas into the surrounding fluid. To address these problems, polymer coatings such as PLGA were used to control Mg degradation, which was demonstrated in a model system using simulated body fluid. Thus, MgO nanoparticles dispersed within polymer composites can be used to enhance bone tissue formation with limited adverse degradation reactions.

In general, HA-containing nanocomposites have been found to exhibit the most suitable mechanical properties for bone tissue applications. The present inventors have found that MgO nanoparticles can be combined with HA nanoparticles (e.g., 10% HA/10% MgO in PLLA) to enhance osteoblast proliferation compared to nanocomposites containing only HA.

The surface topography of pure PLLA and three polymer composites were imaged with SEM, and their surface energy was characterized through contact angle tests. See Example 4 below. Cell adhesion experiments showed that MgO nanoparticles dispersed in PLLA or PLLA/HA composites significantly increased the adhesion of osteoblasts and fibroblasts on PLLA, demonstrating that the composites can be used for regenerating an enthesis. The nanocomposite surface preferably should have an rms surface roughness in the range of 5-300 nm in order to promote cell adhesion and proliferation on the surface of the enthesis regeneration device.

The addition of MgO nanoparticles to HA nanocomposites enhances osteoblast functions and retains the superior mechanical properties of HA nanocomposites for bone applications, with the added benefit of the antibacterial potential of MgO nanoparticles (see Example 6). In principle, any nanoparticulate material capable of releasing Mg²⁺ ions, including Mg phosphate, can be used in place of, or in combination with, MgO nanoparticles.

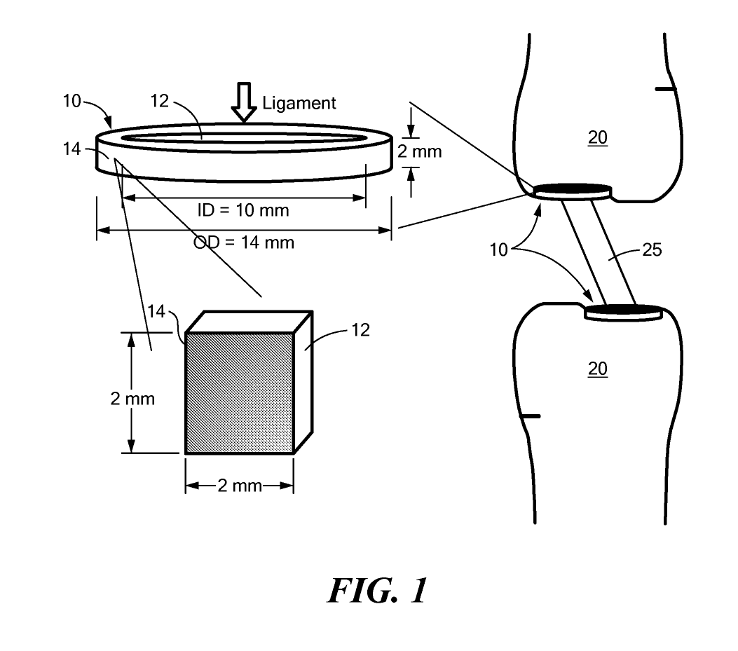

FIG. 1 shows a schematic representation of an embodiment of an enthesis regeneration device (10) fabricated of an orthobiologic material capable of integrating orthopedic soft tissue into hard tissue. The diagram shows the location in the joint where the device (10) is implanted into two bones (20) that articulate at a joint. The ring shape allows the tendon/ligament graft (25) to pass through the center hole of the scaffold to be secured within the bone tunnel with an orthopedic screw. The cutout In the bottom left depicts the optional graded mineralization of the polymer matrix (e.g., PLLA) with mineral nanoparticles (e.g., nanostructured MgO) from a higher concentration at exterior, bone-interfacing surface (14) to a lower concentration at interior, ligament-interfacing surface (12).

The enthesis regeneration device can have any form and size required for reconstructive surgery involving attachment of a tendon, ligament, or artificial graft to bone. Preferably the device Is ring shaped and sized to fit over the tendon, ligament, or graft, and to fit within a prepared cavity at the insertion point in the bone. For example, a device suitable for an anterior cruciate ligament (ACL) graft can have an outer diameter of 14 mm, a height of 2 mm, and an inner diameter of 10 mm. These dimensions can be easily modified to accommodate different patients and even different joints or insertion sites. The device can be used as a biodegradable orthopedic implant for joint reconstructive surgeries to be implanted anywhere the tendon or ligament graft inserts Into bone.

A variety of fabrication methods can be used to produce the device from the nanocomposite material. Such methods include injection molding, extrusion, milling, pouring a melt or suspension into a mold, and wrapping several polymer sheets around one another such that one sheet forms the inner circumference and the final sheet forms the outer circumference. In the wrapping method, the sheets in the middle can contain gradually changing concentrations of nanoparticles so as to provide a gradient of nanoparticle concentration and mineralization. Alternatively, a sheet of nanocomposite material can be made, and ring shapes cut or stamped out from the sheet. In an alternative embodiment, a nanocomposite material of the invention is incorporated into a device, such as a tendon or ligament graft, or scaffold intended to form such a graft, by applying the nanocomposite material as a coating on one or more surfaces of the device. For example, a liquid suspension containing a biodegradable polymer dissolved in a solvent and a plurality of mineral nanoparticles suspended therein can be applied to the device surface, such as by painting, and the solvent removed by evaporation or heating to produce the coated device.

### EXAMPLES

### Example 1

### Synthesis of Nanocrystalline Hydroxyapatite

Following an established procedure, hydroxyapatite (HA) precipitates were synthesized by a wet chemistry process and then treated hydrothermally to produce nanometer-sized HA. See Lopez-Macipe, et al., Wet chemical synthesis of hydroxyapatite particles from nonstoichiometric solutions, J Mater Synth Process, 6, pp. 21-6, 1998; Sato, M., et al., Increased osteoblast functions on undoped and yttrium-doped nanocrystalline hydroxyapatite coatings on titanium, Biomaterials, 27, pp. 2358-69, 2006; and Zhang, L., et al., Biomimetic Helical Rosette Nanotubes and Nanocrystalline Hydroxyapatite Coatings on Titanium for Improving Orthopedic Implants, International Journal of Nanomedicine, 3, pp. 323-34, 2008. With constant stirring, 37.5 mL of a 0.6 M ammonium hydroxide solution was added to 375 mL of deionized water which had been cooled to below 10° C. Approximately 4 mL of ammonium hydroxide was used to adjust the solution pH to about 10. Then, over a period of 12 minutes, 45 mL of a 1 M calcium nitrate solution was slowly (˜3.6 mL/min) dripped into the above mixture while stirring. HA precipitation was observed immediately and was allowed to continue for 10 minutes without stirring. The precipitation reaction is shown in the equation below:

6(NH₄)₂HPO₄+10Ca(NO₃)₂+8NH₄OH→Ca₁₀(PO₄)₆(OH)₂+20NH₄NO₃+6H₂O

Precipitates were centrifuged and rinsed three times before being placed into a 125 mL PTFE-lined acid digestion vessel (Parr Instrument Company) to be treated hydrothermally at 200° C. for 20 hours. Following hydrothermal treatment, the nanometer-sized HA crystals were centrifuged and rinsed with double distilled H₂O, then dried at 80° C. for 12 hours and finally crushed into a nanoscale HA powder using a razor blade for further use.

### Example 2

### Nanoparticle Characterization

A JEOL JEM-101 transmission electron microscope (TEM) was used to characterize the average size and shape of the magnesium oxide (MgO) nanoparticles (purchased from US Research Nanomaterials (20 nm particle size); www.us-nano.com) and HA nanoparticles (synthesized as described in Example 1). The nanoparticle crystal structures were confirmed by x-ray diffraction (XRD), and their chemistry was characterized using Fourier transform infrared spectroscopy (FTIR).

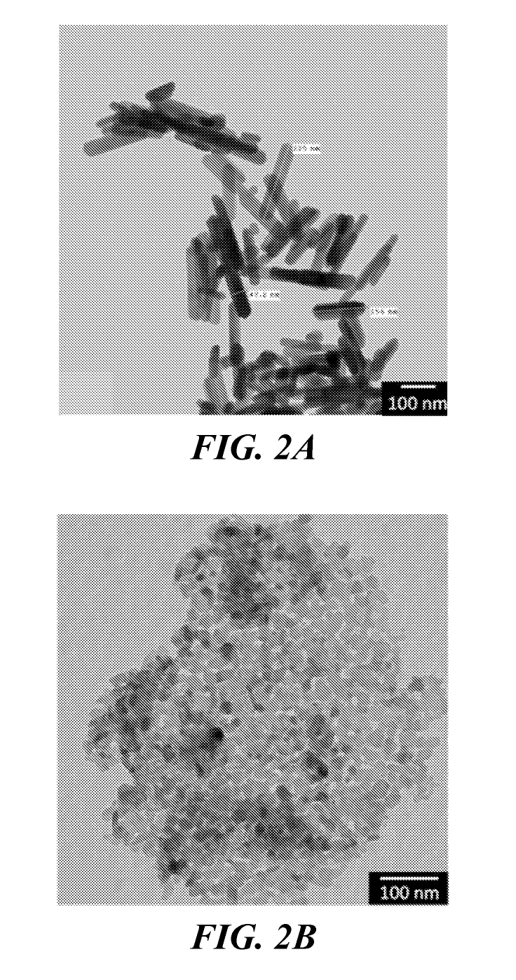

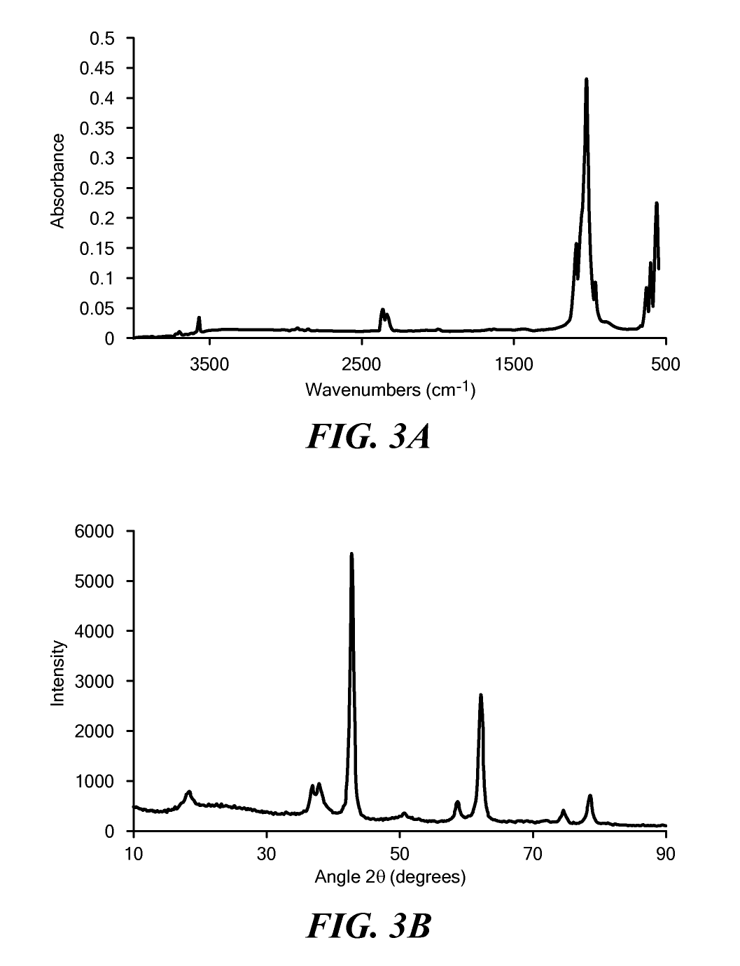

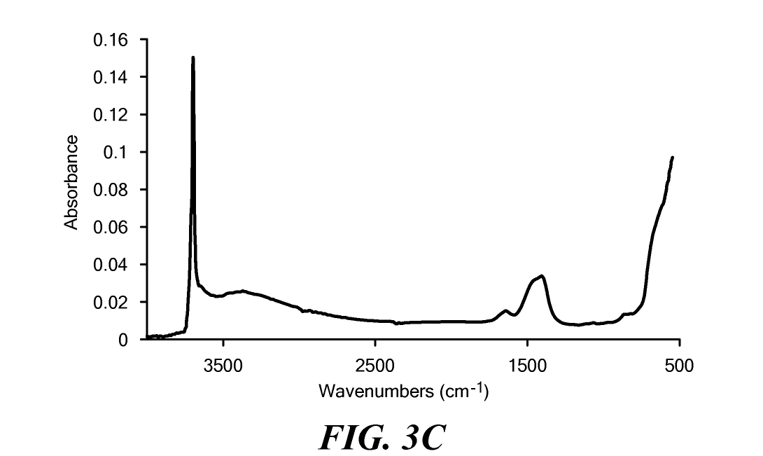

TEM images revealed that the synthesized HA nanoparticles were rod-shaped with an average length of about 200 nm and an average width of about 40 nm (FIG. 2A). MgO nanoparticles appeared circular under TEM, with an average particle diameter of 20 nm (FIG. 2B). The FTIR spectrum, and therefore the chemical composition, of the synthesized nanoparticulate HA matched a spectrum found in literature (FIG. 3A) (Yang B, et al., Preparation and characterization of bone-like hydroxyapatite/poly(methyl methacrylate) composite biomaterials, Science and Engineering of Composite Materials, 20(2), 2013, 147-153). The XRD spectrum of MgO (FIG. 3B) also matched that found in the literature (Li, Z., et al. The synthesis of bamboo structured carbon nanotubes on MgO supported bimetallic Cu—Mo catalysts, DESYGN IT—Special Edition, November 2007). The FTIR spectrum obtained for the nanoparticulate MgO (FIG. 3C) displayed a sharp peak at 3650 cm⁻¹ and a rounded peak around 1450 cm⁻¹. No comparison FTIR spectrum for MgO nanoparticles was found in the literature.

### Example 3

### Preparation of Nanocomposites

Thin polymer nanocomposite sheets were prepared by a casting method. Poly(L-lactic) acid (PLLA) (Polysciences, MW=50,000 Da), HA nanoparticles produced as described in Example 1, and MgO nanoparticles (US Research Nanomaterials, particle size 20 nm) were placed into 20 mL scintillation vials in the amounts indicated in Table 1. Then, 10 mL of chloroform was added to give 3 wt % dry ingredients in chloroform. Each vial was sealed tightly and sonicated at 40 kHz for 1 hour in a water bath set at 55° C., being careful not to exceed the 60° C. boiling temperature of chloroform. After sonication, the suspension appeared homogeneous. The polymer suspensions were poured into 60-mm diameter Pyrex petri dishes and heated at 55° C. for ˜40 minutes to evaporate excess solvent. Samples were then allowed to sit overnight, producing polymeric sheets ˜0.2 mm in thickness.

TABLE 1
Amount (in grams) of dry ingredients used to
produce PLLA nanocomposites

| | PLLA (g) | Nano-HA (g) | Nano-MgO (g) |
|---|---|---|---|
| Plain PLLA | 0.44 | 0 | 0 |
| 20% MgO* | 0.35 | 0 | 0.088 |
| 20% HA | 0.35 | 0.088 | 0 |
| 10% HA/10% MgO | 0.35 | 0.044 | 0.044 |
| 10% MgO | 0.4 | 0 | 0.044 |

*All percentages signify weight percentage of component with respect to combined dry ingredient weight.

### Example 4

### Nanocomposite Surface Characterization

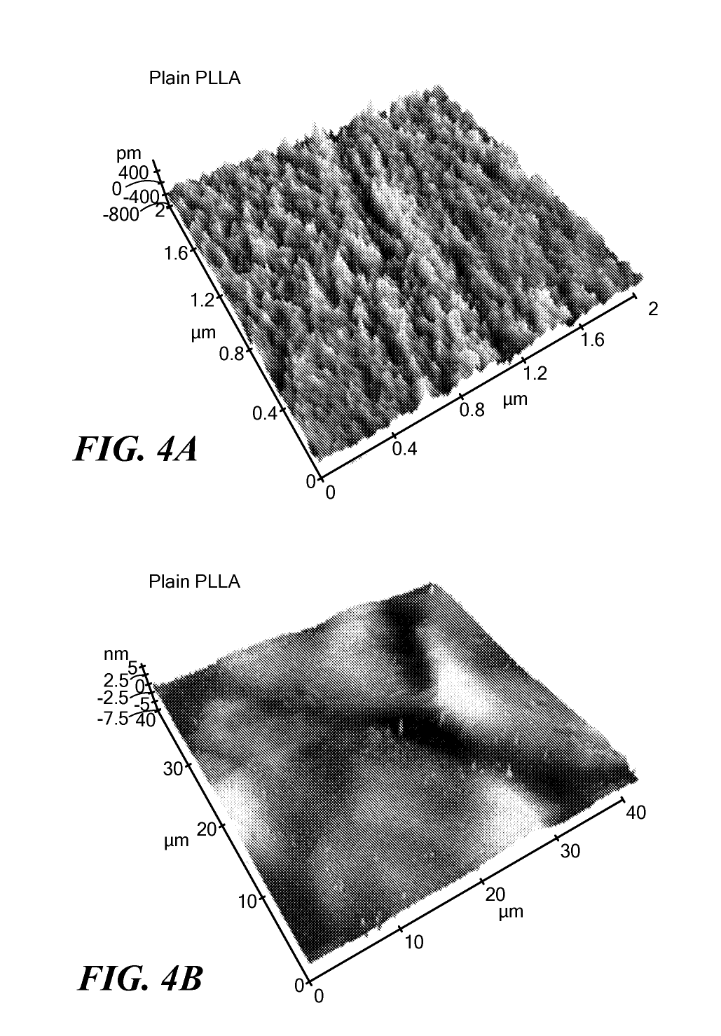

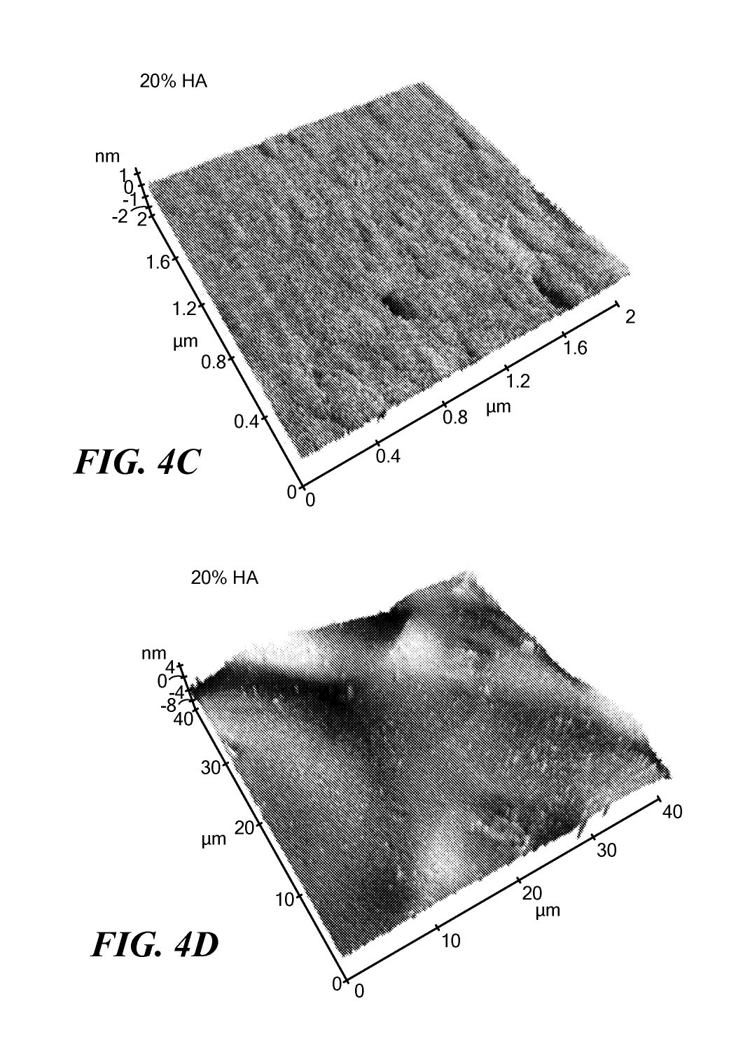

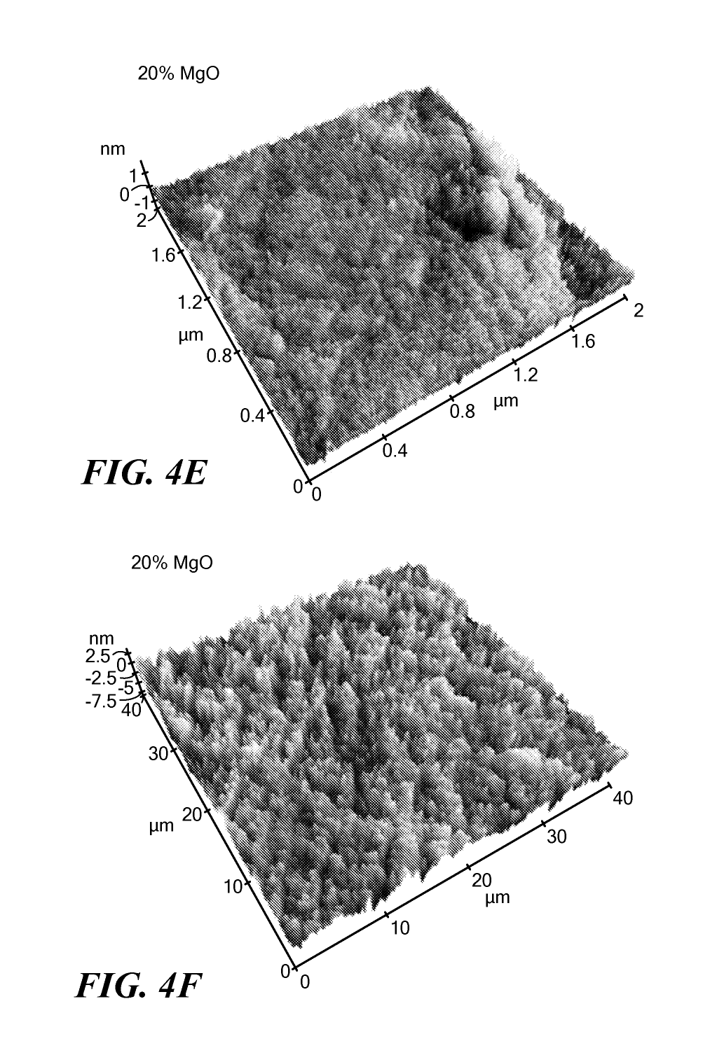

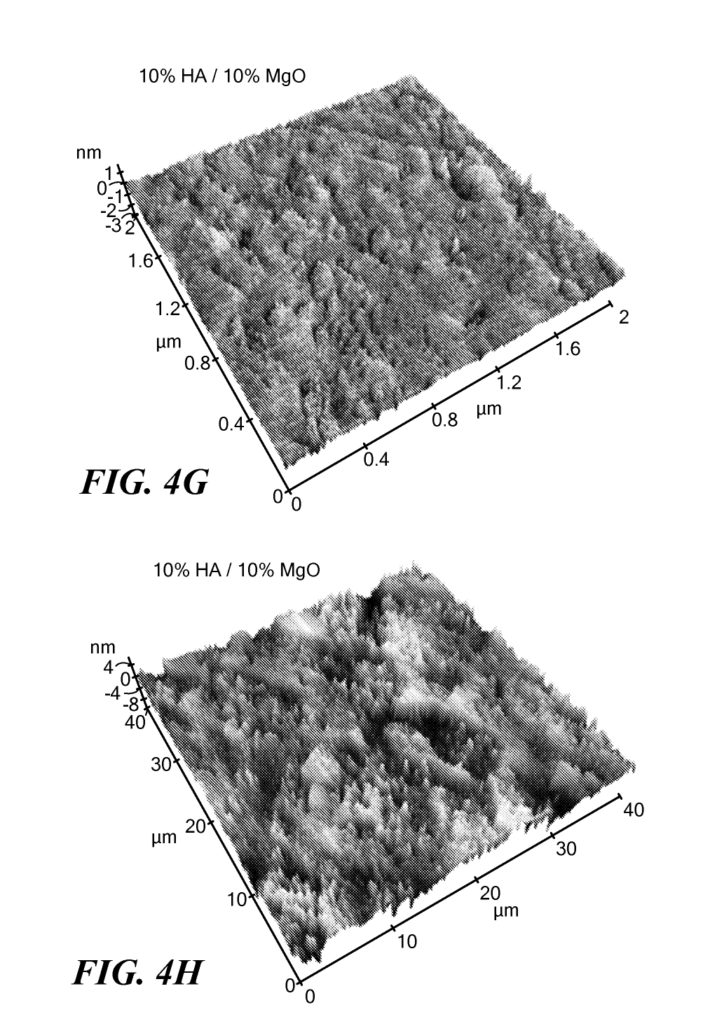

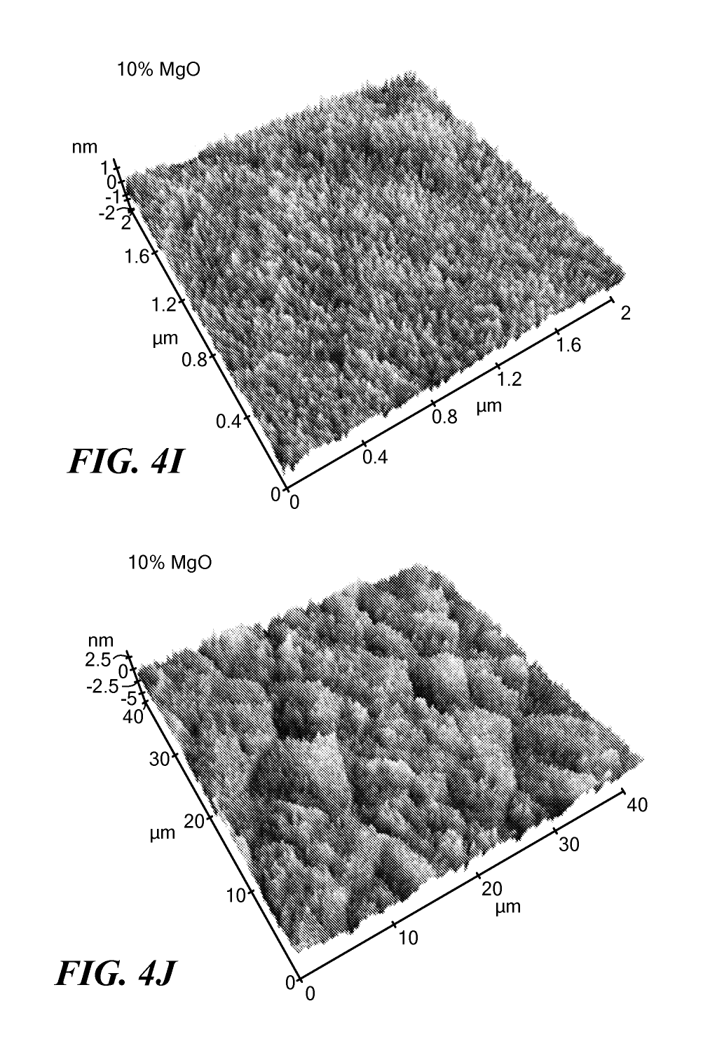

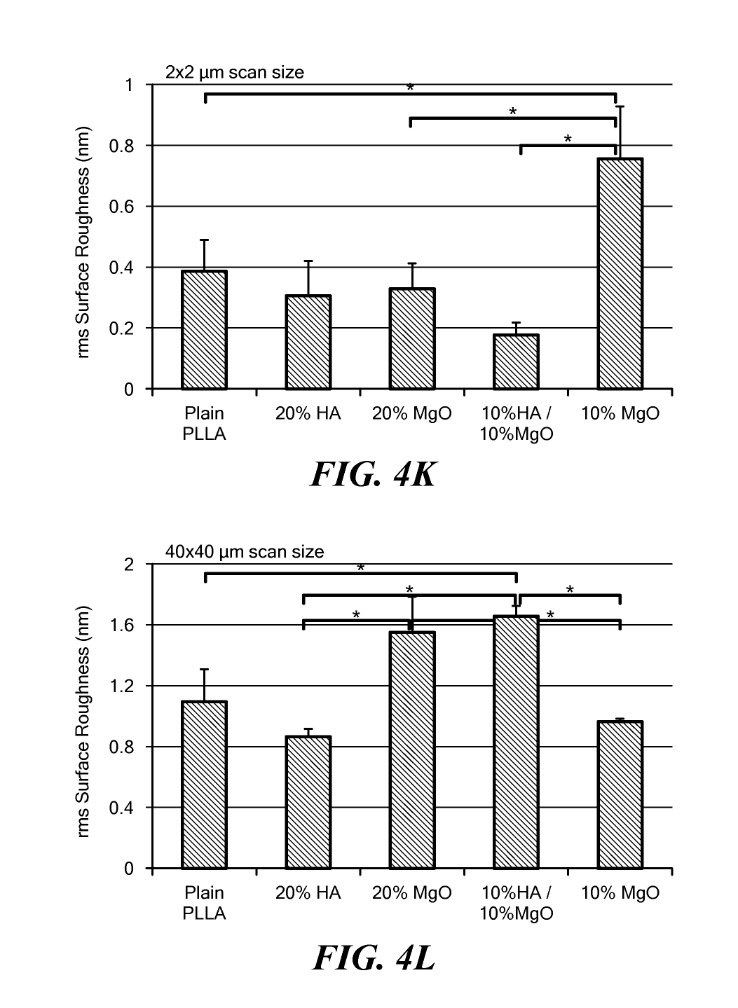

Average surface roughness of the nanocomposites was obtained using a Parks Systems NX-10 atomic force microscope (AFM) with XEI software. A Hitachi S-4800 high resolution field emission scanning electron microscope (SEM) was used to visualize the micro- and nano-topographical features of the nanocomposite surfaces. The results are shown in FIGS. 4A-4L, which show AFM scans of five different samples and their root mean square (rms) surface roughness values at two different scan sizes.

Although there were visible differences in surface texture between samples, the observed surface features had little depth and therefore produced insignificant variations in rms values between samples, with the exception of a 10% MgO sample at 2×2 μm scan size, a 20% MgO sample at 40×40 μm scan size, and a 10% HA 10% MgO sample at 40×40 μm scan size. This result raised the possibility that the nanoparticles dispersed within each nanocomposite sank below the surface of the polymer during curing and were actually covered by a thin layer of PLLA. However, SEM scans of the nanocomposite surfaces (FIGS. 4K and 4L) showed zones where exposed aggregates of nanoparticles could be seen.

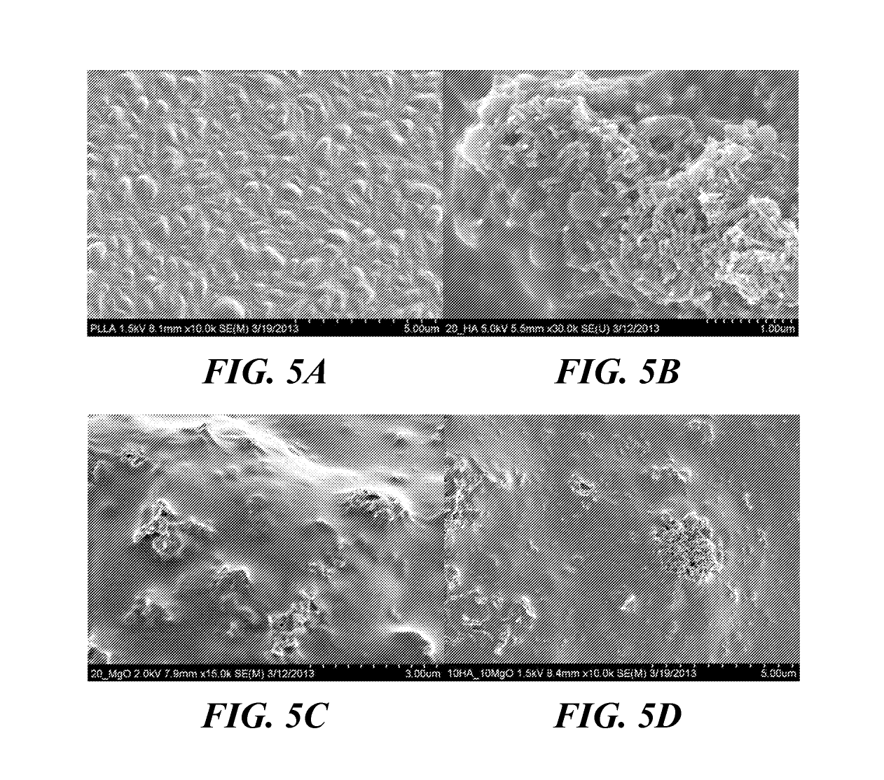

Surface characterization of the nanocomposites by SEM is shown in FIGS. 5A-5D. Some surface structure is present in the PLLA material (FIG. 5A), though the nanocomposites also reveal the presence of HA (FIG. 5B) and/or MgO (FIGS. 5B-5D) nanoparticles at the surface.

The wettability (i.e., hydrophilicity) of PLLA-HA, PLLA-MgO, and PLLA-HA-MgO composite surfaces was examined to assess whether it was altered by the inclusion of HA and/or MgO nanoparticles. Contact angles were measured using a Pioneer 300 Contact Angle Analyzer with accompanying image analysis software. There were no differences in measured contact angles among all samples tested, indicating that the wettability of PLLA was not changed through mineralization with either HA or MgO.

### Example 5

### Mechanical Tensile Testing

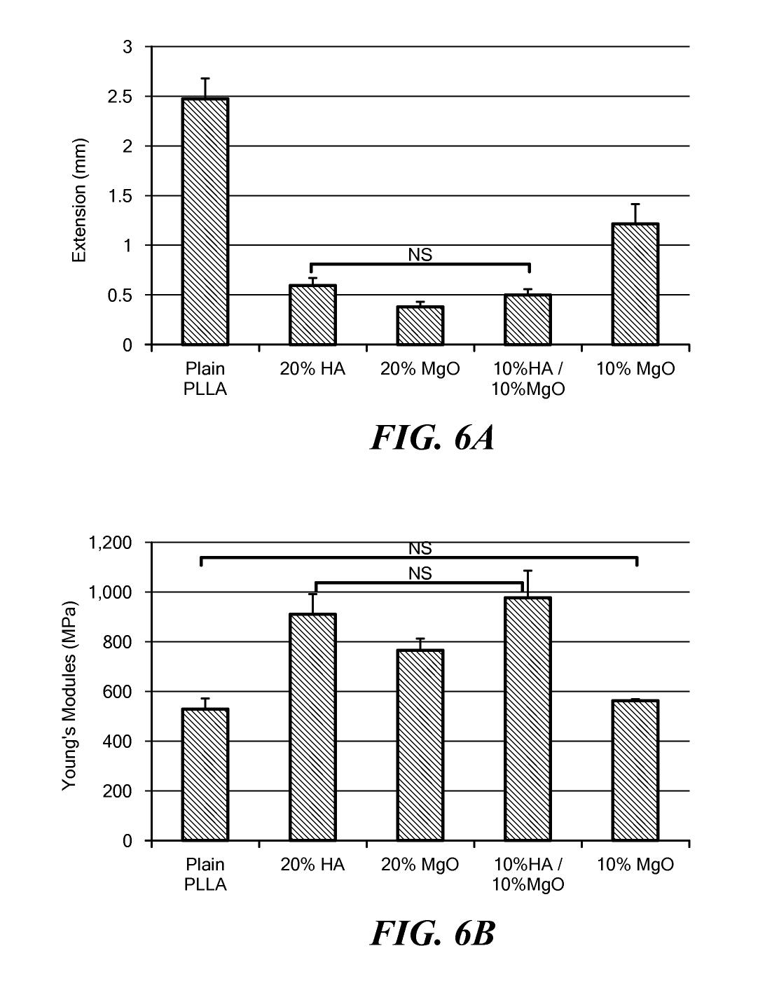

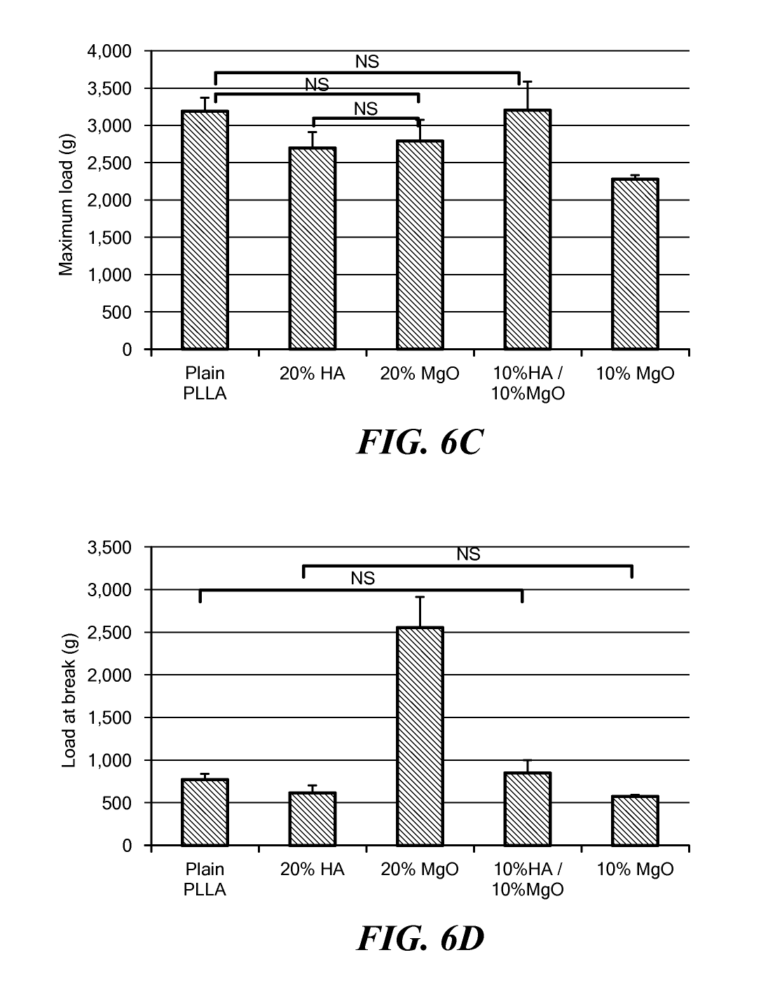

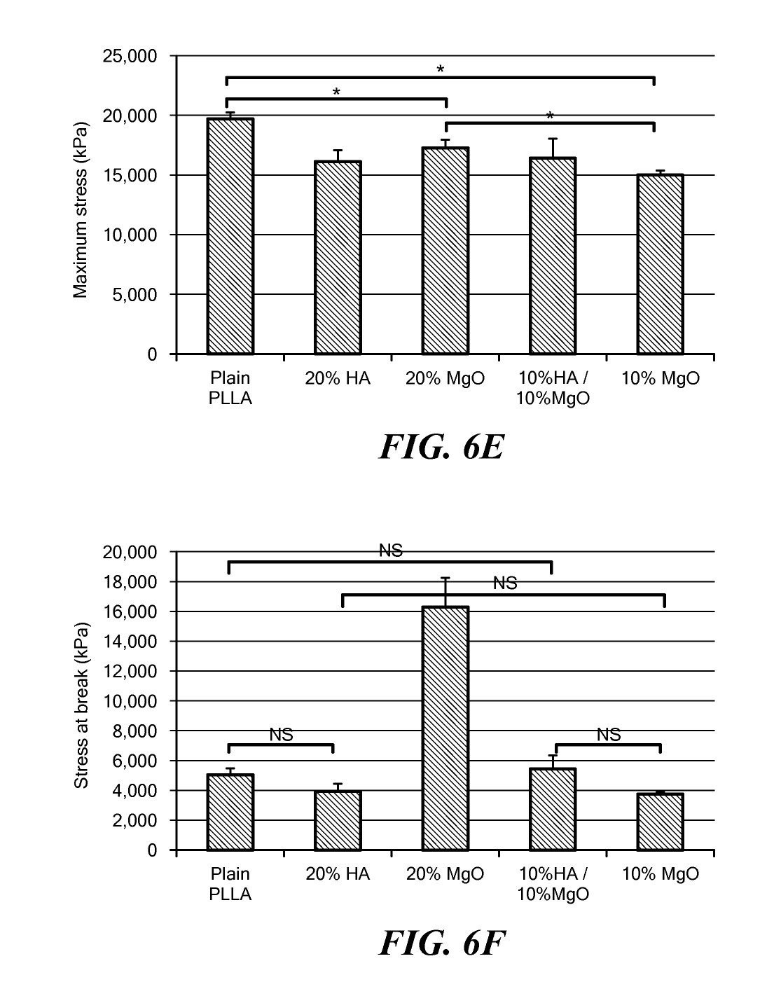

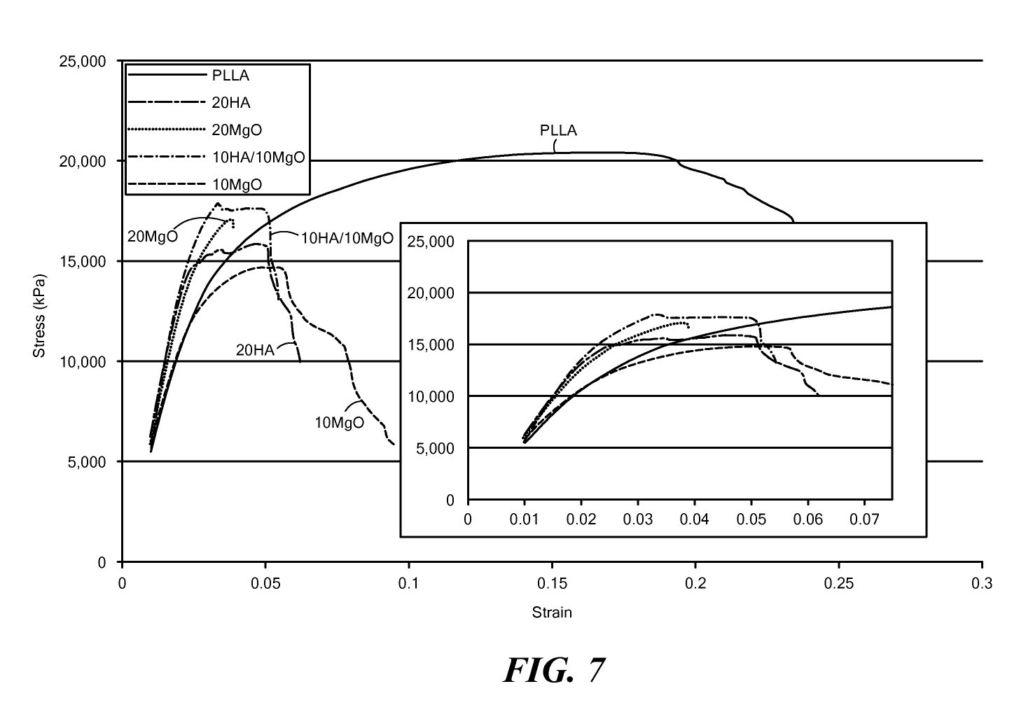

Mechanical tensile testing was performed using a uniaxial tensile tester equipped with a 10 lb load cell and material analysis software (ADMET). Samples were cut into 10 mm×30 mm rectangular strips and secured within the grips of the apparatus such that the initial gauge length was 10 mm. Operating at room temperature with dry samples, the grips were moved apart at a rate of 0.1 mm/s. This arrangement was used to obtain stress-strain curves, as well as the elastic modulus, material elongation, and maximum load endured for each sample. The results are shown in FIGS. 6A-6F and 7.

Mechanical tensile testing revealed that the addition of a secondary nanoparticle phase to plain PLLA stiffened the polymer and increased its elastic modulus. Furthermore, these mechanical properties could be tuned by varying the size, shape, and concentration of nanoparticles in the composite. For instance, the Young's Modulus of plain PLLA did not increase significantly until more than 10 wt % of MgO nanoparticles were added (FIG. 6B). The Young's modulus of tested nanocomposites lay within the range reported for ligament and cancellous bone. Mechanical properties were obtained by uniaxial tensile testing. NS denotes no significant difference (P>0.05) between indicated sample groups. Additionally, the 20% MgO nanocomposite broke upon reaching its maximum sustainable load/stress, whereas the 20% HA nanocomposite, which contained larger, rod-shaped nanoparticles, stretched beyond its maximum load before breaking. This variation in the mode of failure between samples containing HA nanoparticles and MgO nanoparticles was believed due to respective differences in the integration of nanoparticles within the polymer architecture. The smaller, powder-like MgO nanoparticles integrated well within the polymer structure and caused it to break in a chalky, rigid manner, whereas the large, rod-shaped HA nanoparticles remained easily distinguishable as a secondary phase and allowed the PLLA to retain its natural elasticity, leading to a more elastic mode of failure. Plain PLLA, without any added nanoparticles, endured a much more visibly elastic failure, reaching a maximum strain of approximately 25%.

Average stress-strain curves for PLLA and the nanocomposites are presented in FIG. 7. The addition of a secondary nanoparticle phase generally reduced PLLA ductility, but HA nanocomposites generally retained more ductility than MgO nanocomposites.

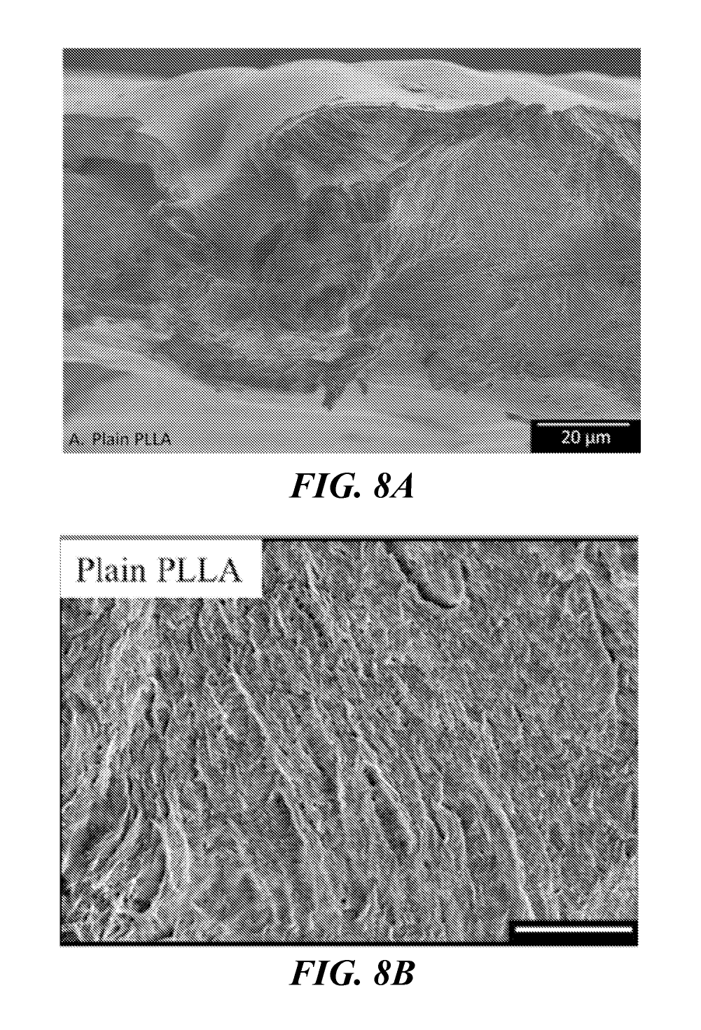

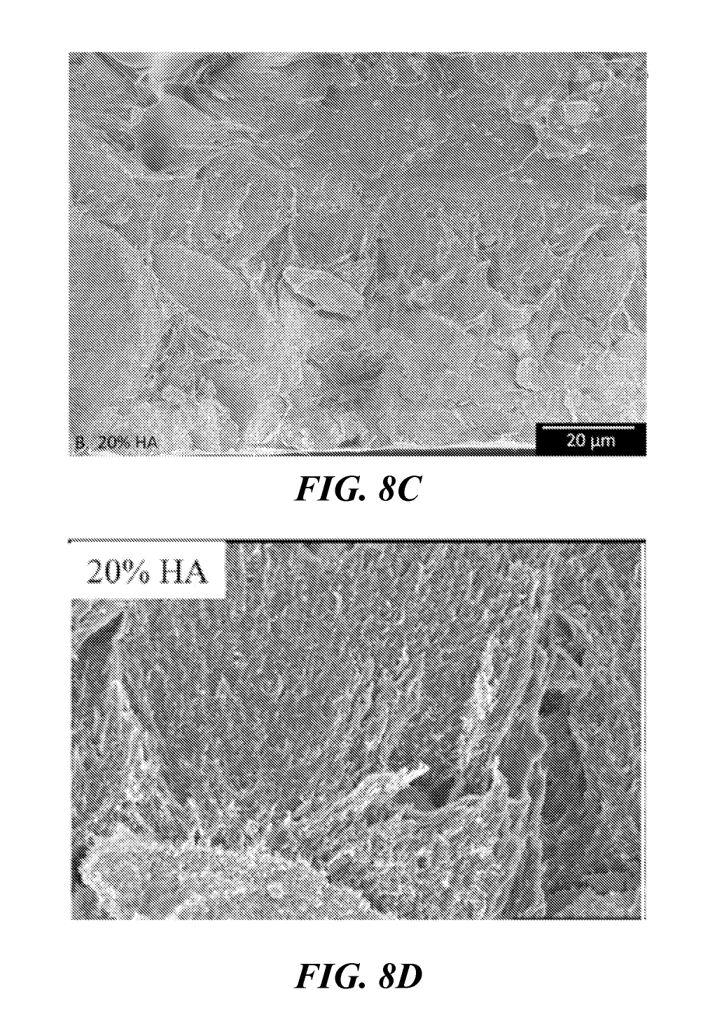

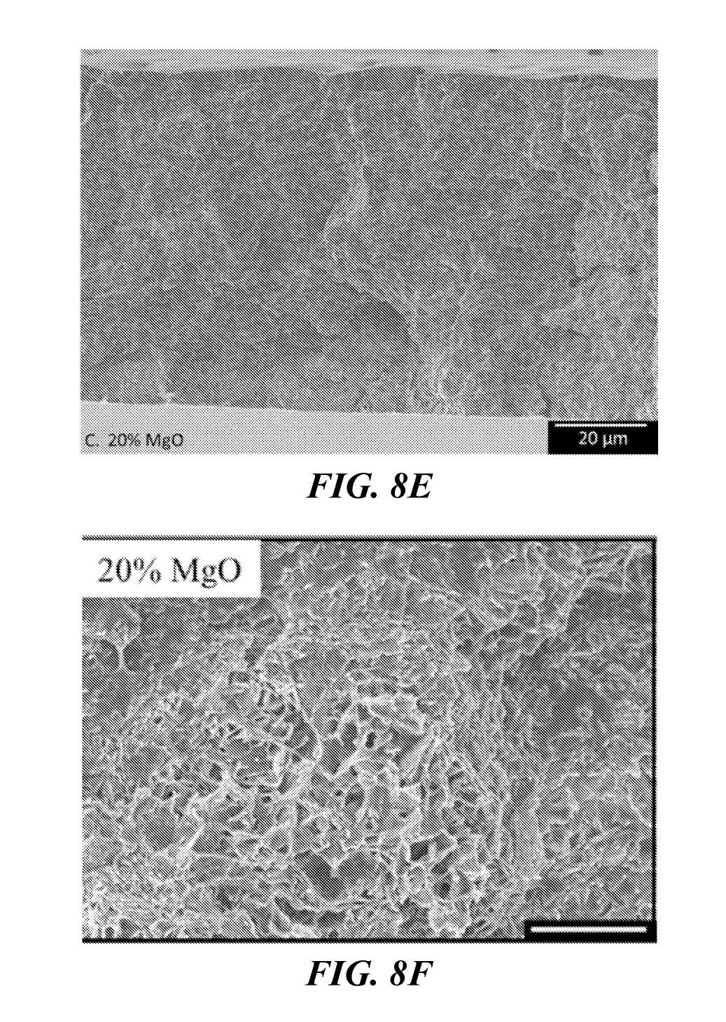

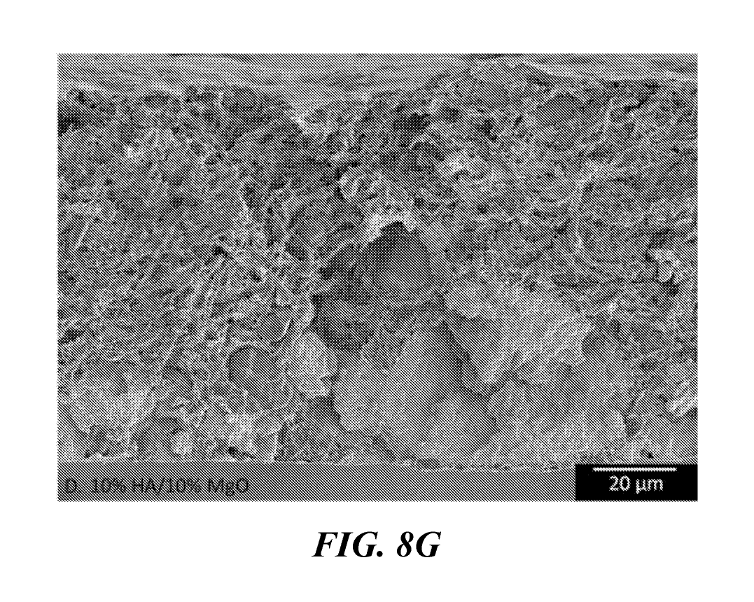

The SEM images of sample failure planes (FIGS. 8A-8D) show considerable differences in internal structure. Samples mineralized with HA retained more elasticity than samples mineralized with MgO. Scale bar=5 μm.

### Example 6

### Antibacterial Properties of Nanoparticles

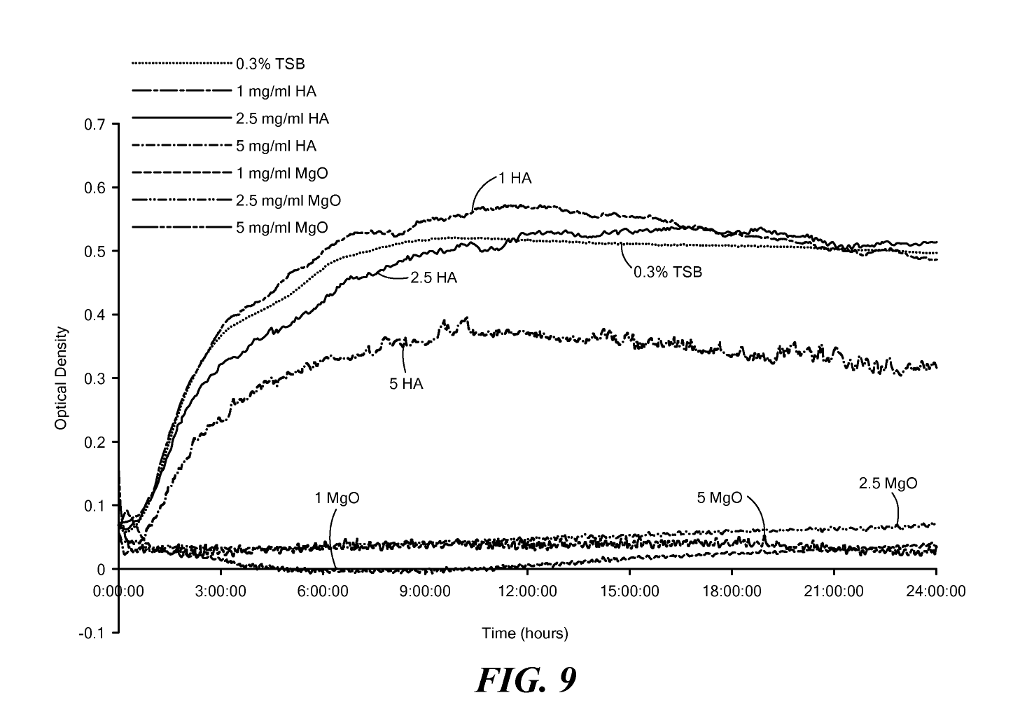

The effects of mineral nanoparticles on bacterial growth are shown in FIG. 9. The growth of Staphylococcus aureus in tryptic soy broth (TSB) with the indicated nanoparticle concentrations was measured via absorbance at 562 nm at 2 min intervals. MgO nanoparticles completely inhibited bacterial growth, whereas HA nanoparticles did not, except for a partial inhibition at the highest HA concentration.

### Example 7

### Cell Adhesion and Proliferation Assays

Primary human osteoblasts (PromoCell, Heidelberg, Germany) were cultured in phenol-free osteoblast basal medium supplemented with osteoblast supplement mix (PromoCell) and 1% penicillin/streptomycin. Primary human dermal fibroblasts (Lonza) were cultured in Dulbecco's modified Eagle medium (DMEM) supplemented with 10% fetal bovine serum (FBS) and 1% penicillin/streptomycin. All cells were cultured to 90% confluence in a 37° C., humidified, 5% CO₂/95% air environment. Cells at passage numbers of 4-12 were used in the experiments.

Polymer nanocomposites were cut into 1 cm×1 cm squares, placed individually into the wells of a 24-well plate, and sterilized under UV light overnight (4 samples were tested from each nanocomposite group for each adhesion trial). Before cell seeding, nanocomposites were rinsed twice with PBS to remove any possible debris. Osteoblasts and fibroblasts were cultured to 90% confluence, rinsed with PBS, and trypsinized using 0.25% trypsin-EDTA (Sigma). Released cells were centrifuged for 3 minutes at 1,200 rpm and then resuspended in their respective media to be counted via a hemacytometer. A solution of 95,000 cells/mL was prepared in cell growth medium, and 1 mL of this solution was added to each well such that initial cell seeding density was 50,000 cells/cm². Samples were incubated under standard culture conditions for 4 hours, then the medium was aspirated from each well and each sample was rinsed with PBS. Samples were transferred to a 24-well plate and 1 mL of cell growth medium was added to each sample along with 200 μL of MTS ((3-(4,5-dimethylthiazol-2-yl)-5-(3-carboxymethoxyphenyl)-2-(4-sulfophenyl)-2H-tetrazolium)) dye. Samples were placed back into the incubator for 4 hours to allow the MTS to completely react with the metabolic products of adherent cells, and then 200 μL of solution from each well was transferred to a 96-well plate in quadruplicate. The 96-well plate was placed into a SpectraMax M3 microplate reader (Molecular Devices) and the absorbance of the MTS solution in each well was measured at a wavelength of 490 nm. The absorbance values of blank wells containing nanocomposite samples without any seeded cells were subtracted from the absorbance values of the corresponding wells containing cells to obtain the absorbance of only the metabolic activity of the cells adherent to each respective nanocomposite sample. The number of cells adherent to each nanocomposite sample was determined by comparing the resulting absorbance values to a standard curve, obtained as described below.

Trypsinized and resuspended cells were counted using a hemacytometer to give a solution of 190,000 cells/ml (190,000 cells/ml/1.9 cm²/well of a 24-well plate=100,000 cells/cm² in a well of a 24-well plate). This solution was added to a 24-well plate in serial dilutions to give 11 different cell densities ranging from 0-100,000 cells/cm². Cells were placed into the incubator under standard culture conditions for 2.5 hours to allow the cells to acclimate to their environment, then 200 μL of MTS dye was added. The 24-well plate was placed back into the incubator for 4 hours to allow the MTS to fully develop and then the solution from each well was transferred to a 96-well plate in quadruplicate. The absorbance of each well was measured at a wavelength of 490 nm to establish a correlation between absorbance value and cell density. The absorbance value of blank wells was subtracted from the absorbance of each cell-containing well to normalize the correlation. A standard curve constructed in this way was prepared for each separate adhesion and proliferation trial.

Cell proliferation assays were conducted with osteoblasts and fibroblasts using the same procedures as the adhesion assays, with the exception that cells were cultured on nanocomposite samples for 1, 3, and 5 days.

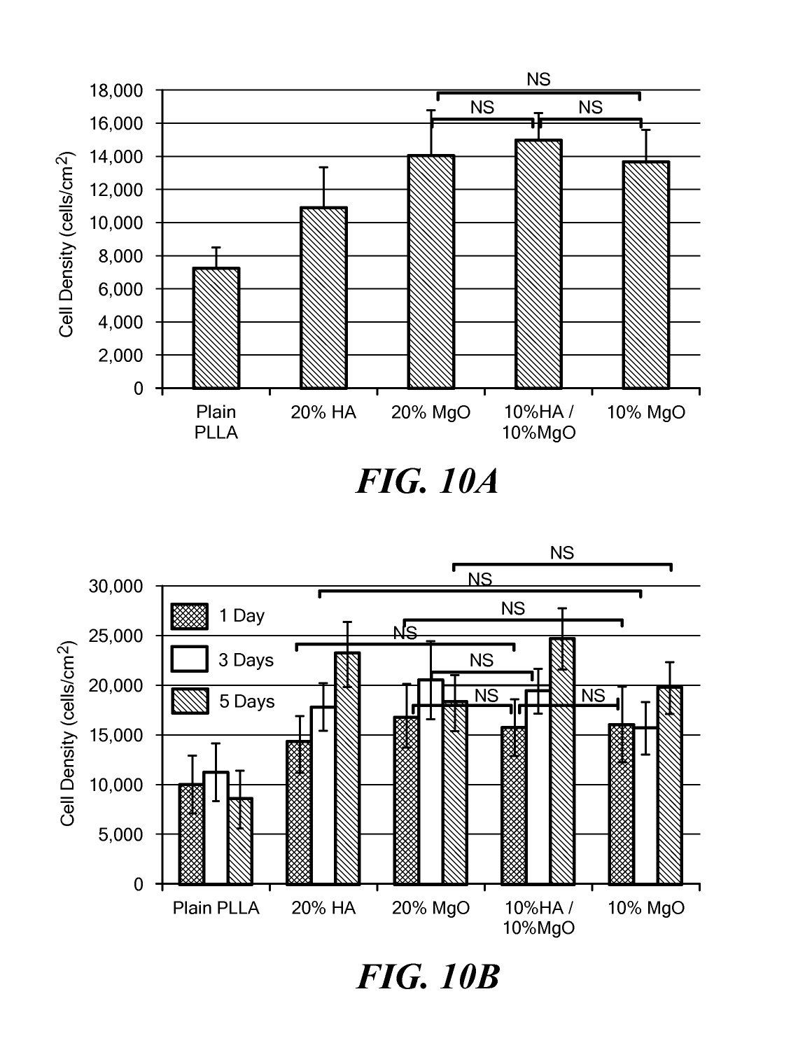

Osteoblasts were found to adhere onto nanocomposite samples containing MgO roughly two times better than onto plain PLLA samples (FIG. 10A). 20% HA samples showed improved adhesion compared to plain PLLA, but fell short of MgO-containing samples (20% MgO, 10% HA/10% MgO, and 10% MgO), showing that the addition of MgO nanoparticles to PLLA enhanced initial osteoblast-PLLA interactions. However, osteoblasts proliferated more rapidly on samples containing HA nanoparticles, and after 5 days of culture, the greatest number of osteoblasts was measured on PLLA nanocomposites containing 10% HA and 10% MgO, followed by PLLA nanocomposites containing 20% HA (FIG. 10B). This demonstrates that MgO nanoparticles can be used in combination with HA nanoparticles to enhance bone cell functions on PLLA. Osteoblast proliferation on plain PLLA was significantly lower compared to all nanocomposite samples at all time points.

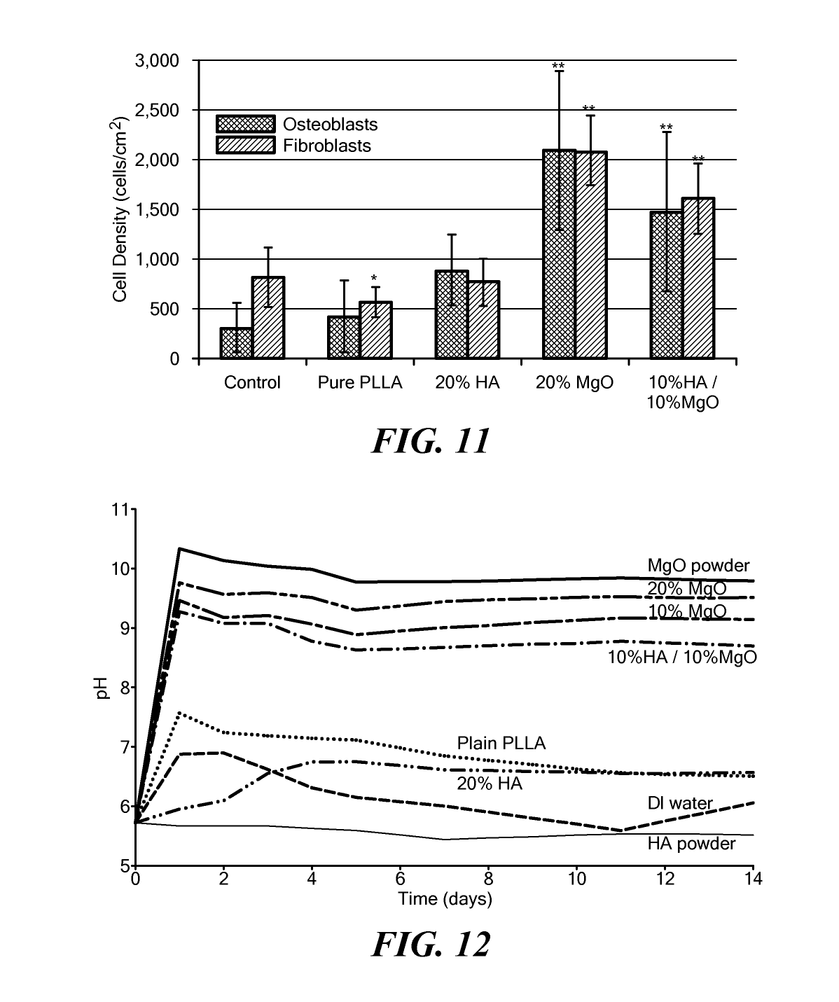

FIG. 11 shows the results of an experiment to measure adhesion of osteoblasts and fibroblasts on polymer composite surfaces. The number of adhered cells at four hours after seeding is represented. The results show that addition of MgO nanoparticles to PLLA significantly Increased osteoblast and fibroblast adhesion. Samples were pure poly(L-lactic acid) (PLLA), PLLA with 20 wt % hydroxyapatite (HA) nanoparticles, PLLA with 20 wt % magnesium oxide (MgO) nanoparticles, and PLLA with 10 wt % HA and 10 wt % MgO. Control was an empty cell culture well. Cell densities were determined using MTT assays and absorbance spectroscopy. Data represents mean±SD, *P<0.05, **P<0.005 compared to control.

Since the surface characterization presented in Example 4 above showed aggregates protruding through the polymer surfaces, the observed differences in osteoblast adhesion and proliferation could be due to a combination of different surface energies, nanotopographies, and chemistries.

### Example 8

### pH Testing

Because the degradation of magnesium into charged species at physiological conditions is a concern for cell well-being, the change in pH of fluid surrounding MgO nanocomposites and nanoparticles was monitored to evaluate the effect that each material has on its immediate environment. Either 30 mg of the nanoparticles of interest or a 1 cm² sample of each nanocomposite variation described above was placed into a 24-well plate containing 2 mL of ultrapure deionized water. The pH of each well was then measured every day for two weeks using a Mettler Toledo Seven Compact Conductivity S230 pH meter.

FIG. 12 shows the change in pH of 2 mL of deionized water containing nanoparticles or a 1 cm² nanocomposite sample. Plain PLLA slowly degraded to generate a more acidic environment, whereas MgO-containing samples produced a more alkaline environment. Water exposed to MgO showed an initial pH increase that leveled off after one day. This alkaline effect of MgO is beneficial to counteract the degradation of PLLA to lactic acid in the body, therefore neutralizing the pH of the fluid surrounding the scaffold. Hydroxyapatite did not cause significant changes in pH.

As used herein, “consisting essentially of” does not exclude materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

1. A composite material comprising a biodegradable polymer matrix, a plurality of MgO nanoparticles embedded in the matrix, and a plurality of hydroxyapatite nanoparticles embedded in the matrix.

2-6. (canceled)

7. The material of claim 1, wherein the hydroxyapatite nanoparticles are present at about 10 wt % to about 60 wt %.

8. The material of claim 7 comprising about 10 wt % MgO nanoparticles.

9. The material of claim 8 comprising about 10 wt % hydroxyapatite nanoparticles.

10. The material of claim 1, further comprising a plurality of adsorbed cells.

11. (canceled)

12. The material of claim 1, wherein the MgO nanoparticles are present in a gradient of concentration through the material.

13. The material of claim 1, wherein the hydroxyapatite nanoparticles are present in a gradient of concentration through the material.

14. The material of claim 1, wherein a surface of the material promotes cell adhesion.

15. (canceled)

16. The material of claim 1, wherein a surface of the material promotes cell proliferation.

17. (canceled)

18. The material of claim 1, further comprising one or more growth factors.

19. The material of claim 18, wherein the material comprises dexamethasone.

20. An enthesis regeneration device comprising the composite material of claim 1 formed into a ring-shaped structure and having an inner surface and an outer surface, the inner surface configured to surround a tendon, ligament, or cell scaffold and the outer surface configured to fit within a bone cavity.

21. The device of claim 20 having an inside ring diameter and an outside ring diameter, wherein the inside diameter is selected to provide contact of the device with said tendon, ligament, or cell scaffold and wherein the outside diameter is selected to provide contact with an inner surface of said bone cavity.

22. The device of claim 21, wherein the ligament is an anterior cruciate ligament.

23. The device of claim 21, wherein the inside diameter is about 10 mm.

24. The device of claim 21, wherein the outside diameter is about 14 mm.

25. The device of claim 21, wherein the ring-shaped structure has a wall thickness of about 2 mm.

26. The device of claim 21, wherein MgO nanoparticles in the material form a gradient from a lower concentration at said inner surface to a higher concentration at said outer surface.

27. (canceled)

28. The device of claim 1, wherein the hydroxyapatite nanoparticles form a gradient from a lower concentration at said inner surface to a higher concentration at said outer surface.

29. The device of claim 28, wherein MgO nanoparticles in the material form a gradient from a lower concentration at said inner surface to a higher concentration at said outer surface.

30. A method of fabricating the composite material of claim 1, the method comprising the steps of:
(a) providing a solution of poly(L-lactic acid) in a solvent;
(b) suspending MgO nanoparticles and hydroxyapatite nanoparticles in the solution to form a suspension;
(c) placing the suspension in a mold; and
(d) removing the solvent to form the composite material.

31. The method of claim 30, wherein the solvent is removed by heating.

32. The method of claim 30, wherein the composite material is produced in sheet form.

33. (canceled)

34. A method of attaching a ligament or tendon to a bone, the method comprising the steps of
(a) attaching the enthesis regeneration device of claim 21 to a detached end of a ligament or tendon, whereby the device surrounds the detached end;
(b) anchoring the detached end of the ligament or tendon within a hole or tunnel in a bone of an animal; and
(c) allowing the device to form a new enthesis between the bone and the ligament or tendon.

35. A method of attaching a soft tissue to a hard tissue, the method comprising the steps of:
(a) attaching a device comprising the composite material of claim 1 to a soft tissue;
(b) anchoring the device and attached soft tissue to a hard tissue; and
(c) allowing cell attachment and proliferation within the device to attach the soft tissue to the hard tissue.

36. A kit comprising the enthesis regeneration device of claim 21 and a container for the device.

37. The kit of claim 36 further comprising one or more tools for attaching the device to a tissue.
